(19) United States
(12) Patent Application Publication
(10) Pub. No.: US 2014/0142628 A1
(43) Pub. Date: May 22, 2014

(54) SURGICAL IMPLANT SYSTEM AND METHOD

(71) Applicant: WARSAW ORTHOPEDIC, INC., Warsaw, IN (US)

(72) Inventors: Vincent C. Traynelis, Chicago, IL (US); Kevin R. Humphreys, Oxford, MS (US); Mark C. Dace, Collierville, TN (US)

(73) Assignee: Warsaw Orthopedic, Inc., Warsaw, IN (US)

(21) Appl. No.: 13/680,250

(22) Filed: Nov. 19, 2012

(51) Int. Cl. A61B 17/84

(52) U.S. Cl. CPC A61B 17/84
USPC 606/246

(57) ABSTRACT

A surgical fastener comprises a head including a wall and a drive interface oriented in a first axial direction. At least one tissue penetrating element extends from the wall in a second axial direction. The at least one element is provisionally disposed with tissue and the wall overlaps a surface of an implant to selectively position and orient the implant relative to tissue. Systems and methods are disclosed.

### TECHNICAL FIELD

The present disclosure generally relates to medical devices for the treatment of musculoskeletal disorders, and more particularly to a surgical system for fastening implants to tissue at a surgical site and a method for treating a spine.

### BACKGROUND

Spinal pathologies and disorders such as scoliosis and other curvature abnormalities, kyphosis, degenerative disc disease, disc herniation, osteoporosis, spondylolisthesis, stenosis, tumor, and fracture may result from factors including trauma, disease and degenerative conditions caused by injury and aging. Spinal disorders typically result in symptoms including deformity, pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes fusion, fixation, discectomy, laminectomy, correction and implantable prosthetics. As part of these surgical treatments, implants such as bone fasteners, plates, connectors and vertebral rods are often used to provide stability to a treated region. These implants can redirect stresses away from a damaged or defective region while healing takes place to restore proper alignment and generally support the vertebral members. For example, rods and plates may be attached via the fasteners to the exterior of one or more vertebral members. This disclosure describes an improvement over these prior art technologies.

### SUMMARY

In one embodiment, in accordance with the principles of the present disclosure, a surgical fastener is provided. The surgical fastener comprises a head including a wall and a drive interface oriented in a first axial direction. At least one tissue penetrating element extends from the wall in a second axial direction. The at least one element is provisionally disposed with tissue and the wall overlaps a surface of an implant to selectively position and orient the implant relative to tissue.

### BRIEF DESCRIPTION OF THE DRAWINGS

The present disclosure will become more readily apparent from the specific description accompanied by the following drawings, in which:

FIG. 1 is a perspective view of a component of one embodiment of a surgical system in accordance with the principles of the present disclosure;

FIG. 2 is a perspective view of components of one embodiment of a surgical system in accordance with the principles of the present disclosure disposed with vertebrae;

FIG. 3 is a perspective view of a component of one embodiment of a surgical system in accordance with the principles of the present disclosure;

FIG. 4 is a perspective view of components of one embodiment of a surgical system in accordance with the principles of the present disclosure; and

FIG. 5 is a perspective view of components of one embodiment of a surgical system in accordance with the principles of the present disclosure.

Like reference numerals indicate similar parts throughout the figures.

### DETAILED DESCRIPTION

The exemplary embodiments of a surgical implant system are discussed in terms of medical devices for the treatment of musculoskeletal disorders and more particularly, in terms of a surgical implant system for delivering and/or fastening implants with a surgical site and a method for treating a spine. In one embodiment, the system includes a surgical fastener, such as, for example, a single or dual pronged temporary staple. In one embodiment, the system includes a fastener configured such that only one penetrating element is required to maintain location and orientation of a surgical plate relative to an anterior vertebral surface. It is envisioned that use of a fastener having one penetrating element eliminates the additional steps required when using traditional pins. In one embodiment, the system is configured to provide selected plate placement and reduce surgical time.

In one embodiment, the system includes a fastener configured to provide two points of bone fixation simultaneously to maintain location and orientation of an implant, such as, for example, a surgical plate such that the fastener is not attached to the implant. This fastener configuration provides increased stability and can be quickly inserted and removed from tissue. In one embodiment, the system includes a fastener having a low profile configuration such that a practitioner has a greater work space to perform a surgical procedure adjacent a surgical site.

In one embodiment, the surgical implant system is configured to be delivered with a single insertion tool. The implant includes a drive instrument interface for engagement with the insertion tool.

It is envisioned that the surgical implant system of the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. It is contemplated that the surgical implant system of the present disclosure may be employed with other osteal and bone related applications, including those associated with diagnostics and therapeutics. It is further contemplated that the disclosed surgical implant system may be alternatively employed in a surgical treatment with a patient in a prone or supine position, and/or employ various surgical approaches to the spine, including anterior, posterior, posterior mid-line, lateral, postero-lateral, and/or antero-lateral approaches, and in other body regions. The surgical implant system of the present disclosure may also be alternatively employed with procedures for treating the lumbar, cervical, thoracic, sacral and pelvic regions of a spinal column. The surgical implant system of the present disclosure may also be used on animals, bone models and other non-living substrates, such as, for example, in training, testing and demonstration.

The surgical implant system of the present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

Further, as used in the specification and including the appended claims, “treating” or “treatment” of a disease or condition refers to performing a procedure that may include administering one or more drugs to a patient (human, normal or otherwise or other mammal), employing implantable devices, and/or employing instruments that treat the disease, such as, for example, microdiscectomy instruments used to remove portions bulging or herniated discs and/or bone spurs, in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

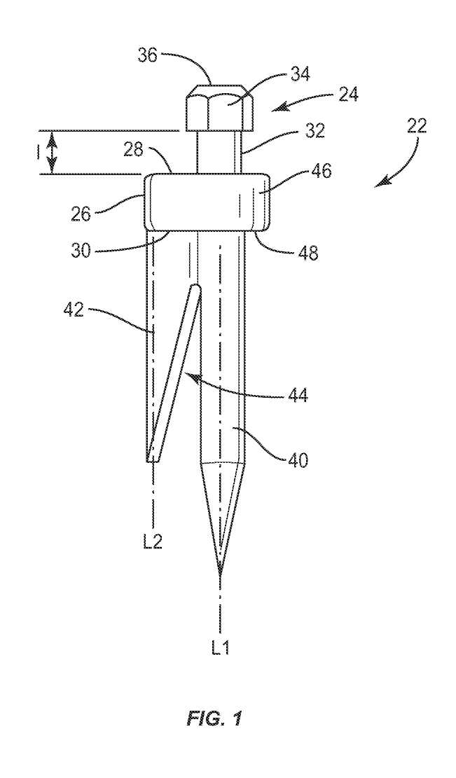

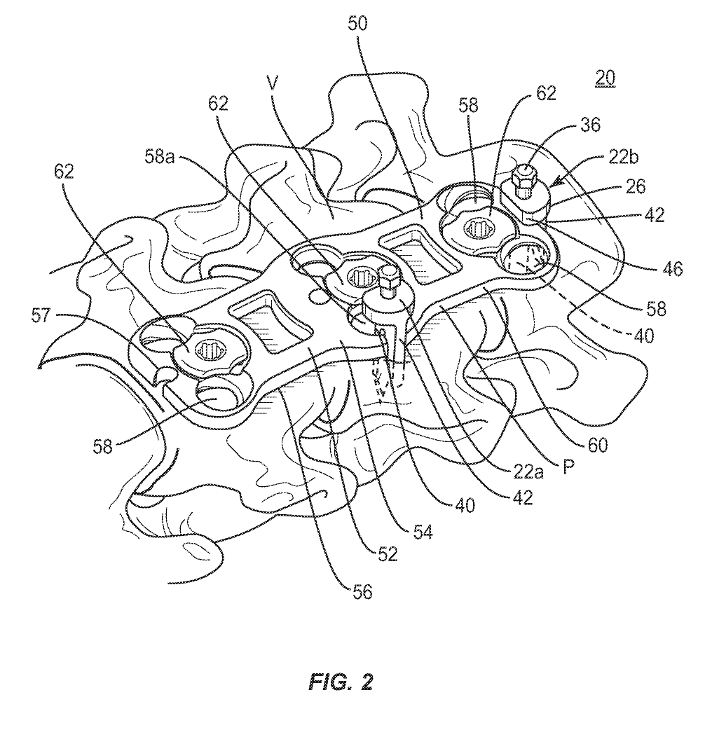

The following discussion includes a description of a surgical implant system, related components and methods of employing the surgical implant system in accordance with the principles of the present disclosure. Alternate embodiments are also disclosed. Reference will now be made in detail to the exemplary embodiments of the present disclosure, which are illustrated in the accompanying figures. Turning now to FIGS. 1 and 2, there are illustrated components of a surgical implant system 20.

The components of surgical implant system 20 can be fabricated from biologically acceptable materials suitable for medical applications, including metals, synthetic polymers, ceramics and bone material and/or their composites, depending on the particular application and/or preference of a medical practitioner. For example, the components of surgical implant system 20, individually or collectively, can be fabricated from materials such as stainless steel alloys, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations. Various components of surgical implant system 20 may have material composites, including the above materials, to achieve various desired characteristics such as strength, rigidity, elasticity, compliance, biomechanical performance, durability and radiolucency or imaging preference. The components of surgical implant system 20, individually or collectively, may also be fabricated from a heterogeneous material such as a combination of two or more of the above-described materials. The components of surgical implant system 20 may be monolithically formed.

Surgical implant system 20 is employed, for example, with an open or mini-open, minimal access and/or minimally invasive including percutaneous surgical technique to deliver and fasten an implant, such as, for example, an anterior cervical plate at a surgical site within a body of a patient, for example, a section of a spine. In one embodiment, the components of surgical implant system 20 are configured to temporarily fix an implantable plate with a provisional fastener to selectively position and orient the plate relative to tissue for a surgical treatment to treat various spine pathologies, such as those described herein. In one embodiment, upon selected positioning and orientation of the plate, permanent fixation elements of system 20 are employed to fix the plate with tissue and the provisional fastener is removed from the tissue. It is contemplated that the provisional fastener can be removed from tissue before or after fixation of the permanent fixation elements. In one embodiment, a permanent fixation element includes components implanted with tissue and remaining after a surgical procedure is completed and an incision is closed. In one embodiment, a permanent fixation element includes components implanted with tissue and remaining after a first and/or initial surgical procedure is completed and an incision is closed, and the component is removable in a second, subsequent and/or separate surgical procedure.

Surgical implant system 20 includes a surgical fastener, such as, for example, a provisional fastener 22. Fastener 22 includes a head 24. Head 24 includes a wall 26 having a first surface 28 and a second surface 30. It is contemplated that surfaces 28, 30 may have surface configurations to enhance engagement such as, for example, rough, arcuate, undulating, dimpled and/or textured, according to the requirements of a particular application. It is envisioned that all or only a portion of wall 26 may have cross section configurations, such as, for example, oval, oblong, triangular, square, polygonal, irregular, uniform, non-uniform, offset, staggered, and/or tapered.

Head 24 includes a stem 32 extending axially from surface 28 of wall 26. Stem 32 includes a surface 34 having a drive interface 36. Drive interface 36 is configured for engagement with a tool, such as, for example a driver. Drive interface 36 is spaced apart from surface 28 by a length L of stem 32. It is envisioned that interface 36 can be alternatively configured, such as, for example, a tri-flat shape having three planar surfaces arranged about an axis of interface 36, square, hexagonal, polygonal, star, groove, Phillips, cruciate, slot or hexalobe cross sectional configuration. It is envisioned that all or only a portion of head 24 may have cross section configurations, such as, for example, hexagonal, oval, oblong, triangular, square, polygonal, irregular, uniform, non-uniform, offset, staggered, and/or tapered depending on a particular application and/or tool.

Fastener 22 includes a first penetrating element, such as, for example, a spike 40. A second penetrating element, such as, for example, a wedge 42. Spike 40 and wedge 42 are connected adjacent surface 30. Spike 40 and wedge 42 extend to respective distal tips, which are disposed in a spaced apart relation. It is contemplated that fastener 22 may include one or a plurality of penetrating elements.

Spike 40 and wedge 42 extend axially from wall 26 in a direction opposite to stem 32. Spike 40 defines a longitudinal axis L1. Wedge 42 defines a longitudinal axis L2. Axis L1 extends from surface 30 in a substantially parallel orientation relative to axis L2. It is contemplated that axes L1, L2 may extend in various relative orientations, such as, for example, transverse, perpendicular and/or other angular orientations such as acute or obtuse. It is contemplated that the penetrating elements can include, such as, for example, barbs, nails or pins. In one embodiment, wedge 42 extends from surface 30 a length to penetrate tissue including bone. In one embodiment, wedge 42 extends from surface 30 a length such that wedge 42 does not penetrate tissue for temporary fixation and is configured to only overlap a portion of an implant, such that only spike 40 penetrates tissue including bone for temporary and/or provisional fixation.

Spike 40 and wedge 42 extend from surface 30 in spaced apart relation to form a cavity 44 therebetween. Cavity 44 is configured for disposal of a portion of an implant, discussed herein. The configuration of wedge 42 is tapered in a decreasing thickness extending from surface 30, such that cavity 44 has a tapered configuration of increasing dimension extending from surface 30. The configuration of cavity 44 has a decreased dimension adjacent surface 30 such that an implant can be temporarily and/or provisionally fixed and retained with the surfaces of spike 40 and wedge 42 for positioning and orientation of the implant. It is contemplated that cavity 44 can be various shapes, such as, for example, oval, oblong, triangular, square, polygonal, irregular, uniform, non-uniform, offset and/or staggered.

Wall 26 includes a transverse flange 46 extending from wall 26. Flange 46 extends transverse to axis L1 and/or axis L2. Flange 46 includes a surface 48 configured to overlap a portion of the implant to provisionally retain and stabilize the implant for positioning and orientation of the implant, as discussed herein. It is contemplated that surface 48 may have surface configurations to enhance engagement such as, for example, rough, arcuate, undulating, dimpled and/or textured, according to the requirements of a particular application.

System 20 includes an implant, such as, for example, an anterior cervical plate 50. Plate 50 includes a stratum 52 having a first surface 54 and a second surface 56. Surface 56 is configured to engage tissue. Stratum 52 defines a series of openings 57 and 58 extending between surface 54 and surface 56. Openings 58 are configured for disposal of permanent implantable fixation elements, such as, for example, bone screws for attaching plate 50 with tissue. Stratum 52 defines an edge 60 disposed around the entire periphery P of plate 50. In one embodiment, plate 50 includes retainers 62 configured to engage fixation elements, such as, for example, bone screws mounted with plate 50. Retainers 62 resist and/or prevent inadvertent backing out of the bone screws after the bone screws are fully inserted into an opening 58 and implanted with tissue.

In one embodiment, as shown in FIG. 2, a fastener 22a includes spike 40 disposed through an opening 58a of plate 50 and penetrating tissue including bone. Wedge 42 extends from surface 30 such that wall 26 overlaps surface 54 and a portion of edge 60 is disposed with cavity 44. Wedge 42 penetrates tissue including bone. Spike 40 and wedge 42 capture edge 60 adjacent surface 30 such that plate 50 can be temporarily and/or provisionally fixed and retained with the surfaces of spike 40 and wedge 42 for selected positioning and orientation of plate 50. The remaining openings 58 of plate 50 are accessible and unobstructed for permanent implantation of fixation elements, such as, for example, bone screws, with tissue to permanently implant plate 50 in a selected position and orientation. In one embodiment, fastener 22a is removable from opening 58a and a bone screw replaces fastener 22a via introduction into opening 58a and permanent implantation with tissue.

In one embodiment, as shown in FIG. 2, a fastener 22b includes flange 46 configured to overlap a portion of edge 60 of plate 50 to provisionally retain and stabilize plate 50 for positioning and orientation of plate 50. Spike 40 and wedge 42 extend exterior to surface 54 and within openings 57, adjacent edge 60, to penetrate tissue including bone. Flange 46 captures edge 60 such that plate 50 can be temporarily and/or provisionally fixed and retained with surface 30 for selected positioning and orientation of plate 50. Openings 58 of plate 50 are accessible and unobstructed for permanent implantation of fixation elements, such as, for example, bone screws, with tissue to permanently implant plate 50 in a selected position and orientation. In one embodiment, fastener 22b is removable from edge 60 and the bone screws and plate 50 are permanently implanted with tissue.

In assembly, operation and use, an implant system, similar to system 20 described herein, is employed with a surgical procedure for treatment of a spinal disorder affecting a section of a spine of a patient, as discussed herein. For example, spinal implant system 20 can be used with a surgical procedure for treatment of a condition or injury of an affected section of the spine including vertebrae. It is contemplated that one or all of the components of system 20 can be delivered or implanted as a pre-assembled device or can be assembled in situ. System 20 may be completely or partially revised, removed or replaced.

For example, system 20 can be employed with a surgical treatment of an applicable condition or injury of an affected section of a spinal column and adjacent areas within a body, such as, for example, cervical vertebrae V, as shown in FIG. 2. It is envisioned that system 20 may be employed with one or a plurality of vertebra. To treat a selected section of vertebrae V, a medical practitioner obtains access to a surgical site including vertebrae V in any appropriate manner, such as through incision and retraction of tissues. It is envisioned that system 20 can be used in any existing surgical method or technique including open surgery, mini-open surgery, minimally invasive surgery and percutaneous surgical implantation, whereby the vertebrae are accessed through a mini-incision, or sleeve that provides a protected passageway to the area. Once access to the surgical site is obtained, the particular surgical procedure can be performed for treating the spine disorder.

An incision is made in the body of a patient and a cutting instrument (not shown) creates a surgical pathway for implantation of components of system 20. A preparation instrument (not shown) can be employed to prepare tissue surfaces of vertebrae V, as well as for aspiration and irrigation of a surgical region according to the requirements of a particular surgical application.

Plate 50 is delivered to the surgical site adjacent vertebrae V. Plate 50 is disposed in a selected position and orientation relative to vertebrae V such that surface 56 of plate 50 is disposed with tissue. Fasteners 22 are delivered to the surgical site adjacent vertebrae V in a configuration to temporarily fix plate 50 with vertebrae V in the selected position and orientation.

A tool (not shown) is utilized to engage drive interface 36 and drive fastener 22a into engagement with plate 50 and penetration with tissue. Spike 40 passes through opening 58a of plate 50 and penetrates tissue including bone of vertebrae V. Wedge 42 extends from surface 30 such that wall 26 overlaps surface 54 and a portion of edge 60 is disposed with cavity 44. Wedge 42 penetrates tissue including bone of vertebrae V. Spike 40 and wedge 42 capture edge 60 adjacent surface 30 to temporarily and/or provisionally fix and retain plate 50 with the surfaces of spike 40 and wedge 42 in the selected position and orientation of plate 50. In one embodiment, fasteners 22 provide two points of temporary and/or provisional bone fixation simultaneously to maintain location and orientation of plate 50. In one embodiment, fasteners 22 have a low profile configuration such that a practitioner has a greater work space to perform a surgical procedure adjacent a surgical site.

The tool is employed to engage drive interface 36 of fastener 22b and drive fastener 22b into engagement with plate 50 and penetration with tissue. Spike 40 passes through opening 57 of plate 50 and penetrates tissue including the base of vertebrae V. Flange 46 overlaps a portion of edge 60 of plate 50 to provisionally retain and stabilize plate 50 in the selected position and orientation of plate 50. Spike 40 and wedge 42 extend exterior to surface 54 and within openings 57, adjacent edge 60, to penetrate tissue including bone of vertebrae V. Flange 46 captures edge 60 such that plate 50 is temporarily and/or provisionally fixed and retained with surface 30 in the selected position and orientation of plate 50.

Pilot holes or the like are made in selected vertebra of vertebrae V corresponding to openings 58 for receiving fixation elements, such as, for example, bone screws (not shown). The bone screws are disposed adjacent vertebrae V at a surgical site and the components of system 20 including a driver, are manipulable to drive, torque, insert or otherwise connect the bone screws with vertebrae V for fastening plate 50 with vertebrae V, according to the particular requirements of the surgical treatment. Bone screws are fully inserted and permanently implanted through openings 58 of plate 50 with tissue to permanently implant plate 50 in the selected position and orientation. Fastener 22a is removed from opening 58a and tissue, and a bone screw replaces fastener 22a via introduction into opening 58a and permanent implantation with tissue including bone of vertebrae V. Fastener 22b is removed from plate 50 and tissue.

Upon completion of the procedure, fasteners 22, surgical instruments and assemblies are removed from the surgical site and the incision is closed. System 20 can be made of radiolucent materials such as polymers. Radiomarkers may be included for identification under x-ray, fluoroscopy, CT or other imaging techniques. It is envisioned that the use of microsurgical and image guided technologies may be employed to access, view and repair spinal deterioration or damage, with the aid of system 20.

It is contemplated one or a plurality of fixation elements may be employed with a single vertebral level. It is further contemplated that the fixation elements may be engaged with vertebrae in various orientations, such as, for example, series, parallel, offset, staggered and/or alternate vertebral levels. It is envisioned that the fixation elements may include one or a plurality of anchors, tissue penetrating screws, conventional screws, expanding screws, wedges, anchors, buttons, clips, snaps, friction fittings, compressive fittings, expanding rivets, staples, nails, adhesives, posts, fixation plates and/or posts. These fixation elements may be coated with an osteoinductive or osteoconductive material to enhance fixation, and/or include one or a plurality of therapeutic agents.

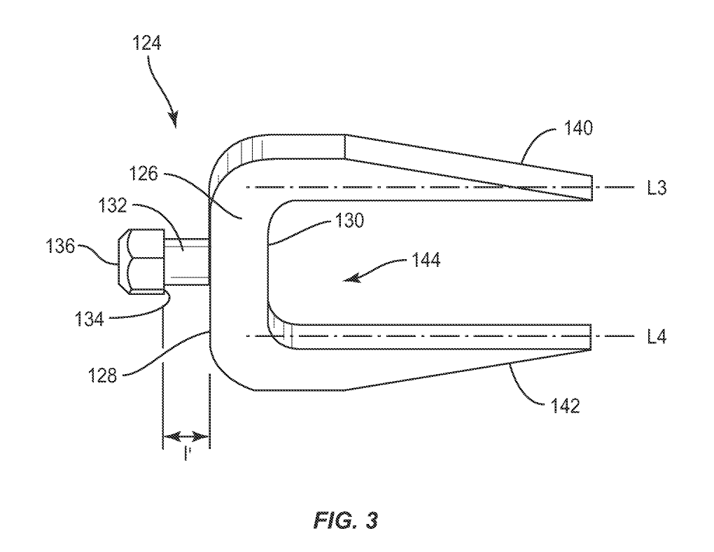

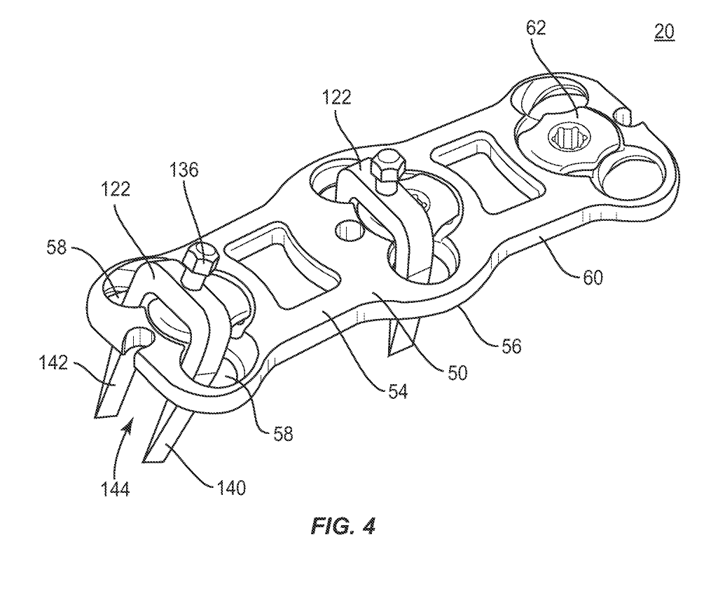

In one embodiment, as shown in FIGS. 3 and 4, surgical implant system 20, similar to the systems and methods described with regard to FIGS. 1 and 2, includes a surgical fastener, such as, for example, a provisional fastener 122. Fastener 122 includes a head 124. Head 124 includes a wall 126 having a first surface 128 and a second surface 130. Head 124 includes a stem 132 extending axially from surface 128 of wall 126. Stem 132 includes a surface 134 having a drive interface 136 configured for engagement with a tool, such as, for example a driver. Drive interface 136 is spaced apart from surface 128 by a length L5 of stem 132. It is envisioned that interface 136 can be alternatively configured, similar to those alternatives described above. It is envisioned that all or only a portion of head 124 may have cross section configurations, such as, for example, hexagonal, oval, oblong, triangular, square, polygonal, irregular, uniform, non-uniform, offset, staggered, and/or tapered depending on a particular application and/or tool.

Fastener 122 includes a staple configuration and has penetrating elements extending axially from surface 130 of wall 126. Fastener 122 includes a first penetrating element, such as, for example, a first prong 140 and a second penetrating element, such as, for example, a second prong 142. Prongs 140, 142 are connected by surface 130. Prongs 140, 142 extend to respective distal tips, which are disposed in spaced apart relation. It is contemplated that fastener 122 may include one or a plurality of penetrating elements.

Prong 140 defines a longitudinal axis L3. Prong 142 defines a longitudinal axis L4. Axis L3 extends from surface 130 in a substantially parallel orientation relative to axis L4. It is contemplated that axes L3, L4 may extend in various relative orientations, such as, for example, transverse, perpendicular and/or other angular orientations such as acute or obtuse. It is contemplated that the penetrating elements have an outer surface that can include an engagement element, such as, for example, barbs, spikes, rough surfaces, notches, tabs, ribs, nails or pins to enhance provisional fixation of fastener 122 in tissue.

Prong 140 and prong 142 are spaced apart along surface 130 of wall 126 and form a cavity 144. Cavity 144 is configured for disposal of a portion of an implant, discussed herein. Cavity 144 has a substantially rectangular configuration. It is contemplated that cavity 144 can be various shapes, such as, for example, oval, oblong, triangular, square, polygonal, irregular, uniform, tapered, non-uniform, offset and/or staggered.

Fasteners 122 are oriented such that prongs 140, 142 are disposed through openings 58 of plate 50, described above, and penetrating tissue including bone, as shown in FIG. 4. Surface 130 overlaps surface 54 such that a portion of plate 50 is disposed with cavity 144. Prongs 140, 142 capture surface 54 adjacent surface 130 such that plate 50 can be temporarily and/or provisionally fixed and retained with the surfaces of prongs 140, 142 and surface 130 for selected positioning and orientation of plate 50. The remaining openings 58 of plate 50 are accessible and unobstructed for permanent implantation of fixation elements, such as, for example, bone screws, with tissue to permanently implant plate 50 in a selected position and orientation. In one embodiment, fasteners 122 are removable from openings 58 and bone screws replace fasteners 122 via introduction into openings 58 and permanent implantation with tissue. In one embodiment, fastener 122 may include one of prongs 140, 142 disposed with an opening 58 and the other of prongs 140, 142 outside of surface 54 such that prongs 140, 142 and surface 130 capture edge 60 adjacent surface 130 to temporarily and/or provisionally fix and retain plate 50, similar to that described above.

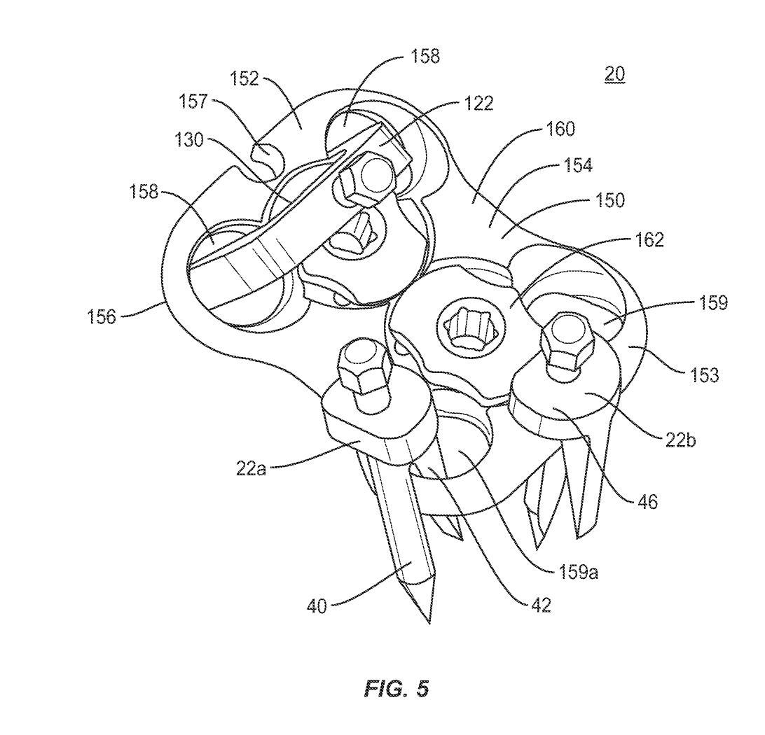

In one embodiment, as shown in FIG. 5, surgical implant system 20, similar to the systems and methods described with regard to FIGS. 1-4, includes an implant, such as, for example, an anterior cervical plate 150 having an arcuate and/or angled configuration to conform to the orientation of vertebrae. It is contemplated that plate 150 may be employed to achieve lordosis or sagittal alignment of vertebrae.

Plate 150 includes a first portion 152 and a second portion 153. In one embodiment, portion 153 extends in an arcuate orientation from portion 152. In one embodiment, portion 153 extends at an angular orientation from portion 152.

Plate 150 has a first surface 154 and a second surface 156. Surface 156 is configured to engage tissue. Portion 152 defines a first pair of openings 158 and portion 153 defines a second pair of openings 159. Openings 158, 159 are configured for disposal of permanent implantable fixation elements, such as, for example, bone screws for attaching plate 150 with tissue. Plate 150 defines an edge 160 disposed around an entire periphery of plate 150. In one embodiment, plate 150 includes retainers 162 configured to engage fixation elements, such as, for example, bone screws mounted with plate 150. Retainers 162 resist and/or prevent inadvertent backing out of the bone screws after the bone screws are fully inserted into openings 158, 159 and implanted with tissue.

Spike 40 of fastener 22a, described above, passes through opening 159a of plate 150 and penetrates tissue including bone. Wedge 42 extends from surface 30 such that wall 26 overlaps surface 154 and a portion of edge 160 is disposed with cavity 44. Wedge 42 penetrates tissue including bone. Spike 40 and wedge 42 capture edge 160 adjacent surface 30 to temporarily and/or provisionally fix and retain plate 150 with the surfaces of spike 40 and wedge 42 in a selected position and orientation of plate 150.

Flange 46 of fastener 22b, described above, overlaps a portion of edge 160 of plate 150 to provisionally retain and stabilize plate 150 in the selected position and orientation of plate 150. Spike 40 and wedge 42 extend exterior to surface 154 and within openings 157, adjacent edge 160, to penetrate tissue including bone. Flange 46 captures edge 160 such that plate 150 is temporarily and/or provisionally fixed and retained with surface 30 in the selected position and orientation of plate 50.

Fasteners 122, described above, are oriented such that prongs 140, 142 are disposed through openings 158 of plate 150 and penetrating tissue including bone. Surface 130 overlaps surface 154 such that a portion of plate 150 is disposed with cavity 144. Prongs 140, 142 capture surface 154 adjacent surface 130 such that plate 150 can be temporarily and/or provisionally fixed and retained with the surfaces of prongs 140, 142 and surface 130 for selected positioning and orientation of plate 150. The remaining opening 159 of plate 150 is accessible and unobstructed for permanent implantation of a fixation element, such as, for example, a bone screw, with tissue to permanently implant plate 150 in the selected position and orientation. Fasteners 122 and 22a are removable from openings 158, 159a, respectively, and bone screws replace fasteners 122 and 22a via introduction into openings 158, 159a and permanent implantation with tissue. Fastener 22b is removed from plate 150 and tissue.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

1. A surgical fastener comprising:
a head including a wall and a drive interface oriented in a first axial direction, the drive interface being fixed relative to the wall; and
at least one tissue penetrating element extending from the wall in a second axial direction,
wherein the at least one element is configured to be provisionally disposed with tissue such that the wall overlaps a surface of an implant to selectively position and orient the implant relative to tissue.

2. A surgical fastener as recited in claim 1, wherein the at least one tissue penetrating element includes a first element and a second element spaced apart from the first element.

3. A surgical fastener as recited in claim 1, wherein the at least one tissue penetrating element includes a staple having a first element and a second element.

4. A surgical fastener as recited in claim 1, wherein the at least one tissue penetrating element includes a spike and a wedge spaced apart from the spike.

5. (canceled)

6. A surgical fastener as recited in claim 1, wherein the at least one tissue penetrating element and the wall define a tapered cavity configured for disposal of a portion of the implant.

7. A surgical fastener as recited in claim 1, wherein the wall includes a transverse flange configured to overlap the surface of the implant.

8. A surgical fastener as recited in claim 1, wherein the at least one tissue penetrating element is configured for disposal within an opening defined by the implant.

9. A surgical fastener as recited in claim 1, wherein the at least one tissue penetrating element includes a first element and a second element spaced apart from the first element, the first element being configured for disposal within a first opening of the implant and the second element being configured for disposal within a second opening of the implant.

10. A surgical implant system comprising:
at least one fastener comprising a wall and a drive interface, and at least one tissue penetrating element extending from the wall, the drive interface being fixed relative to the wall; and
a plate defining at least one opening and including a first surface and a second surface oriented to engage tissue,
wherein the at least one element is configured to be provisionally disposed with the at least one opening and tissue such that the wall overlaps the first surface to selectively position and orient the plate relative to tissue.

11. A surgical implant system as recited in claim 10, wherein the at least one tissue penetrating element includes a staple having first element and a second element.

12. A surgical implant system as recited in claim 10, wherein the at least one tissue penetrating element includes a spike and a wedge spaced apart from the spike.

13. A surgical implant system as recited in claim 10, wherein the at least one tissue penetrating element and the wall define a cavity configured for disposal of a portion of the plate.

14. A surgical implant system as recited in claim 10, wherein the at least one tissue penetrating element and the wall define a tapered cavity configured for disposal of a portion of the plate.

15. A surgical implant system as recited in claim 10, wherein the at least one tissue penetrating element includes a first element and a second element spaced apart from the first element, the first element being configured for disposal within a first opening of the plate and the second element being configured for disposal within a second opening of the plate.

16. A surgical implant system as recited in claim 10, wherein the at least one fastener includes a first fastener such that the at least one tissue penetrating element includes a staple having first element and a second element and a second fastener such that the at least one tissue penetrating element includes a spike and a wedge spaced apart from the spike.

17. A surgical implant system as recited in claim 16, wherein the at least one opening of the plate includes a first pair of openings and a second opening, the elements of the first fastener being disposed with the first pair of openings and the spike being disposed with the second opening.

18. A method for treating a spine, the method comprising the steps of:
providing at least one fastener comprising a wall and a drive interface, and at least one tissue penetrating element extending from the wall;
providing an implant including a first surface and a second surface oriented to engage tissue;
provisionally fastening the at least one fastener with tissue such that the at least one tissue penetrating element is disposed with tissue and the wall overlaps the first surface to selectively position and orient the implant relative to tissue;
fixing the implant with the tissue via fixation elements corresponding to the selected position and orientation; and
removing the at least one fastener from the tissue.

19. A method as recited in claim 18, wherein the at least one tissue penetrating element includes a staple having first element and a second element, and the tissue includes at least one cervical vertebrae.

20. A method as recited in claim 18, wherein the implant includes a cervical plate defining at least one opening configured for disposal of the at least one penetrating element.

21. A surgical fastener as recited in claim 4, wherein the wedge is fixed relative to the spike, the spike having a maximum length that is greater than that of the wedge.
